(19) United States
(12) Patent Application Publication
(10) Pub. No.: US 2009/0110752 A1
(43) Pub. Date: Apr. 30, 2009

(54) COMPOSITION FOR CONTROLLING BLOOD GLUCOSE AND METHOD THEREOF

(75) Inventors: Huey-Fang Shang, Taipei (TW); Ting-Kai Leung, Taipei (TW); Yung-Sheng Lin, Hsinchu (TW); Chung-Huei Hsu, Taipei (TW)

Correspondence Address: VOLPE AND KOENIG, P.C., UNITED PLAZA, SUITE 1600, 30 SOUTH 17TH STREET, PHILADELPHIA, PA, 19103, US

(73) Assignees: TAIPEI MEDICAL UNIVERSITY, Taipei (TW); NATIONAL APPLIED RESEARCH LABORATORIES, Taipei (TW)

(21) Appl. No.: 12/028,044

(22) Filed: Feb. 8, 2008

(30) Foreign Application Priority Data
Oct. 29, 2007 (TW) 096140638

(51) Int. Cl. A61K 33/26; A61K 33/08; A61K 47/30; A61P 3/10; A61P 3/08

(52) U.S. Cl. 424/647; 424/691; 514/772

(57) ABSTRACT

A composition for controlling a blood glucose and a method thereof are provided. The composition comprises a far-infrared ray releasing substance, wherein the composition reduces the blood glucose of a subject via an irradiation of the far-infrared ray releasing substance. In another aspect, the method comprises the steps of providing a far-infrared ray releasing substance having an oxide mineral as a main component, and disposing the far-infrared ray releasing substance in a place close to a subject in an appropriate distance, wherein the subject has an insulin resistance and the appropriate distance is within an irradiation range of the far infrared ray.

### FIELD OF THE INVENTION

The present invention relates to a composition and a method for controlling blood glucose, and more particularly to a far-infrared releasing substance and the method for controlling blood glucose of a patient who has insulin resistance.

### BACKGROUND OF THE INVENTION

The patients who have non-insulin dependent diabetes mellitus or type II diabetes take about 90-95 percent among all diabetic patients. The dominant syndrome of the type II diabetes is insulin resistance, which results from the insulin cannot be used by the cells of the patients, and thus excessive insulin is produced. Since the insulin in the patient who has type II diabetes cannot bring its normal effects, the glucose in the blood keeps at a high level and will cause many complications over a long period of time, such as the cardiovascular diseases, the ophthalmic diseases and the renal diseases.

Generally, the oral hypoglycemic drug or the insulin injection should be used in cooperation with the diet control in order to control the blood glucose in a normal or near-normal level. However, the oral hypoglycemic drugs will loose its effect gradually with the course of the diabetes. Moreover, the insulin therapy may not have a remarkable effect on all diabetic patients. Therefore, a more effective way is required for controlling the blood glucose.

According to International Commission on Illumination (CIE1987), the far-infrared ray (FIR) is an electromagnetic wave with the wavelength of 3-1000 μm. Among them, the far-infrared ray having the wavelength of 4-16 μm is called the light of life, because of its advantages in the growth of animals and plants. Currently, it has been proved that FIR has therapeutic effect on many human diseases, and thus is often applied to many physiological purposes. However, the effect on controlling the blood glucose by the far-infrared ray has not been investigated.

In view of the drawbacks of current techniques, the inventors develop a composition and a method for controlling the blood glucose by a far-infrared ray releasing substance. The irradiation of the far infrared ray is a physical property, which prevents the side effects of the oral drugs. Further, the far-infrared ray releasing substance has a carriable advantage that can control the blood glucose continually and overcome the drawback of short-term effect of the insulin therapy. The summary of the present invention is described below.

### SUMMARY OF THE INVENTION

It is an aspect of the present invention to provide a pharmaceutical composition for controlling a blood glucose of a subject having an insulin resistance, comprising a far-infrared ray releasing substance including an 80%-99.9% oxide mineral in weight, wherein the oxide mineral comprises a 60-95% aluminum oxide in weight. The pharmaceutical composition is not only carriable and easy to be used, but also normalizes the blood glucose continually via the far-infrared ray irradiation. Particularly, the far-infrared ray releasing substance will not release free irradiation, and it also has negative ion that is beneficial to the human body. Hence, it will not cause undesirable side effects to the human body.

Preferably, the pharmaceutical composition further comprises a pharmaceutically acceptable carrier.

Preferably, the insulin resistance comprises one selected from a group consisting of a type II diabetes, a post-surgical syndrome, a serious illness, a burn injury, a post-traumatic syndrome, a related complication and a combination thereof.

Preferably, the far-infrared ray releasing substance irradiates a far-infrared ray with an emissivity over 0.9 at a wavelength in a range of 4-16 μm.

Preferably, the far-infrared ray releasing substance reduces the blood glucose at room temperature.

Preferably, the far-infrared ray releasing substance further comprises a 1-20% ferric oxide, a 1-10% magnesium oxide and a 1-30% calcium carbonate in weight.

It is another aspect of the present invention to provide a composition for controlling a blood glucose of a subject, comprising a far-infrared ray releasing substance wherein the composition reduces the blood glucose of the subject via an irradiation of the far-infrared ray releasing substance.

According to the present invention, the subject has an insulin resistance.

Preferably, the far-infrared ray releasing substance comprises an oxide mineral, for example, an aluminum oxide.

In one preferred embodiment, the composition is a pharmaceutical composition. Preferably, the pharmaceutical composition further comprises a pharmaceutically acceptable carrier.

In another preferred embodiment, the composition further comprises a base material mixed with the far-infrared releasing substance.

Preferably, the base material is at least one selected from a group consisting of a metal, a glass, a ceramic and a polymer.

It is a further aspect of the present invention to provide a method for controlling a blood glucose. In an exemplary embodiment, the method comprises the steps of providing a far-infrared ray releasing substance and disposing the far-infrared ray releasing substance in a place close to a subject in an appropriate distance, wherein the subject has an insulin resistance and the appropriate distance is within an irradiation range of the far infrared ray.

Preferably, the far-infrared ray releasing substance irradiates the far-infrared ray with an emissivity over 0.9 at a wavelength in a range of 4-16 μm, and reduces the blood glucose of the subject via the far-infrared ray irradiation.

Preferably, the far-infrared ray releasing substance comprises an oxide mineral, for example, an aluminum oxide.

Preferably, the far-infrared ray releasing substance further comprises a 1-20% ferric oxide, a 1-10% magnesium oxide and a 1-30% calcium carbonate in weight.

Preferably, the insulin resistance comprises one selected from a group consisting of a type II diabetes, a post-surgical syndrome, a serious illness, a burn injury, a post-traumatic syndrome, a related complication and a combination thereof.

Other objects, advantages and efficacies of the present invention will be described in detail below taken from the preferred embodiments with reference to the accompanying drawings, in which:

### BRIEF DESCRIPTION OF THE DRAWINGS

FIG. 1 is a diagram showing the results of the blood glucose of the control group and the FIR group mice according to a preferred embodiment of the present invention, wherein the upper line represents the blood glucose of the control group mice, and the lower line represents that of the FIR group mice.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

### Example I

### The Far-Infrared Ray (FIR) Releasing Substance

The FIR releasing substance of the present invention is a high-efficiency far-infrared ray ceramic powder developed by the Instrument Technology Research Center of the National Applied Research Laboratories and the Taipei Medical University. The biological effect of the FIR releasing substance is proved by many experiments, and the composition thereof is composed of various natural minerals. These natural minerals, mainly the oxide minerals, in the FIR releasing substance are about 80-99.9% in weight, and comprise 60-95% aluminum oxide, 1-20% ferric oxide, 1-10% magnesium oxide and 1-30% calcium carbonate. Besides, the FIR releasing substance further comprises other ingredients including titanium dioxide, titanium boride and more natural minerals such as silicon oxide, zinc hydroxide, zinc oxide and carbides, etc. The average emissivity of the FIR releasing substance is over 0.9 at a wavelength between 4-16 μm, which is measured by an FIR spectrometer using a black body as a standard. Furthermore, the far-infrared ray released by the FIR releasing substance has an anti-bacterial rate of over 99.9% against Staphylococcus and Escherichia coli according to the AATCC100 standard.

### Example II

### The Far-Infrared Ray (FIR) Pharmaceutical Composition for Controlling the Blood Glucose

A pharmaceutical composition for controlling the blood glucose of an insulin-resistant subject is provided in the present invention, which comprises a far-infrared ray (FIR) releasing substance, and the components and the properties of the FIR releasing substance are the same as those in Example I. It is noticed that the insulin resistance herein represents the disease resulting from both genetic factors and non-genetic factors, for example, the type R diabetes, the insulin resistance following a surgery, a serious illness, a burn injury, a trauma, a related complication and a combination thereof. In order to be applied to the subject via different routes, the pharmaceutical composition further comprises a pharmaceutical acceptable carrier such as the excipient, the adhesive and the assistant agent. According to the present invention, the composition normalizes the blood glucose of the insulin-resistant subject to a lower level via an irradiation of the far-infrared ray releasing substance. Additionally, since the pharmaceutical composition herein has the advantages of reducing the blood glucose at room temperature and being carriable, it provides an alternative choice for the subject who has insulin resistance, besides the oral drugs and the intravenous injection.

### Example III

### The Far-Infrared Ray (FIR) Composition

The present invention relates to a composition for controlling the blood glucose of a subject based on the third preferred embodiment, which comprises an FIR releasing substance and reduces the blood glucose of the subject via an irradiation of the far-infrared ray releasing substance. The components and the properties of the FIR releasing substance are the same as those in Example I and not further described here. In the present embodiment, the subject has an insulin resistance including the type II diabetes, the insulin resistance following a surgery, a serious illness, a burn injury, a trauma, a related complication and a combination thereof. However, the composition is not only a pharmaceutical composition; it can further be mixed with or distributed on a base material to carry out its effect through different ways. According to the present invention, the base material is at least one selected from a group consisting of a metal, a glass, a ceramic and a polymer. For example, when the FIR releasing substance is mixed with a tile, it serves as a building material for reducing the blood glucose of the subject, and the blood glucose of the subject can be controlled once the subject is surrounded with the environment built by the building material. Probably, the FIR releasing substance is mixed with a polymer and serves as a cloth for reducing the blood glucose. It means that the subject carries the FIR releasing substance with the blood glucose reducing effect upon putting on the cloth.

### Example IV

### The Effect of the FIR Releasing Substance on Normalizing Blood Glucose of Burned Mice

Multiple studies have documented that hyperglycemia and insulin resistance are consequences of burn injury and associated with increased morbidity and mortality. This situation also widely occurs on critically ill, post-surgical and traumatic patients. In this embodiment, a burned mice model is used to prove the effect of the composition of the present invention on controlling blood glucose.

7-week old BALB/c male mice are randomly separated into the control group (13 mice) and the FIR group (13 mice). The mice are anesthetized and their backs are shaved the day before the experiment. Then, a fire prevention, heat insulation board with a 30×25 mm window is attached to the shaved position of the mice, and a 0.3 ml 95% alcohol is dropped on the exposed surface followed by lighting a fire in order to burn the surface of the mice for 15 seconds. The above procedure will cause 30% burn injury on the surface of the mice, and the mice are supplemented with 1 ml physiological saline immediately via intraperitoneal injection. The two groups of mice are placed separately, wherein the FIR group is placed around the environment containing the FIR ceramic powder within the irradiation range of the far infrared ray. In the 24 hours following burn injury, the mice are on fasting but provided with drinking water continually. After 24 hours, the food and drink for the mice are restored. On the second, the fourth, the seventh and the fourteenth days after burn injury, the mice are sacrificed separately for measuring the amount of blood glucose.

### RESULT

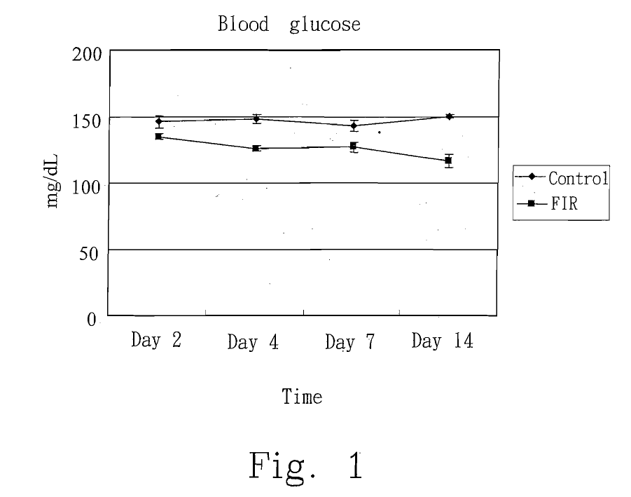

Please refer to FIG. 1 and Table 1, wherein Table 1 shows the records of the blood glucose of the mice measured on the second, the fourth, the seventh and the fourteenth days after burn injury. In each measurement, 3 mice per group are sacrificed for obtaining the values of the blood glucose. On the fourteenth day, the control group contains blood glucose of two mice since the other two mice have been dead, whereas the FIR group contains that of four mice. FIG. 1 is a diagram showing the results of Table 1, wherein each dot represents the average blood glucose of the mice on that day. The upper line represents the blood glucose of the control group, and the lower line represents that of the FIR group. As FIG. 1 illustrates, the blood glucose of the control group mice does not change apparently; however, the blood glucose of the FIR group mice has a descending trend with time. The result reveals that the FIR group mice get a more stable control over their blood glucose than the control group, and the difference thereof reaches the level of statistical significance (p<0.05, as Table 2 illustrates). Additionally, as to the mortality, a mouse is dead on the first day and another mouse is dead on the second day after burn injury in the control group, while the survival rate of the FIR group reaches 100%. Therefore, it is further proved that the FIR releasing substance of the present invention improves the survival rate after burn injury. By using the burn injury model to simulate the insulin resistance, it is found that the FIR releasing eases the problem of high blood glucose efficiently and thus makes the blood glucose get better control. In the future, the composition comprising the FIR releasing substance can be applied to the diseases with an insulin resistance, such as the type II diabetes, in order to normalize the blood glucose of the patients and provide an alternative choice besides the oral hypoglycemic drugs and the insulin therapy.

TABLE 1

| | | FIR |
|---|---|---|
| Date | Control group (mg/dL) | group (mg/dL) |
| 2^{nd} day (three mice) | 148, 141, 149 (146 ± 4.4) | 135, 137, 133 |
| | | (135 ± 2.0) |
| 4^{th} day (three mice) | 152, 145, 147 (148 ± 3.6) | 124, 126, 128 |
| | | (126 ± 2.0) |
| 7^{th} day (three mice) | 144, 146, 138 (142.7 ± 4.2) | 126, 131, 124 |
| | | (127 ± 3.6) |
| 14^{th} day (two mice in the | 151, 149 (150 ± 1.4) | 110, 121, |
| control group, 4 mice in the | | 119, 116 |
| FIR group) | | (116.5 ± 4.8) |

TABLE 2

| Burn injury | Control group | FIR group |
|---|---|---|
| Average | 146.3636 | 125.3846 |
| Variation | 17.65455 | 59.42308 |
| Number of observed values | 11 | 13 |
| Pooled variation | 40.43738 | |
| Hypothesized mean difference | 0 | |
| Degree of freedom | 22 | |
| T test | 8.05297 | |
| P(T <= t) one-tailed | 2.64 × 10⁻⁸ | |
| Critical value: one-tailed | 1.717144 | |
| P(T <= t) two-tailed | 5.29 × 10⁻⁸ | |
| Critical value: two-tailed | 2.073875 | |

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A pharmaceutical composition for controlling a blood glucose of a subject having an insulin resistance, comprising:
a far-infrared ray releasing substance including an 80%-99.9% oxide mineral in weight, wherein the oxide mineral comprises a 60-95% aluminum oxide in weight.

2. A pharmaceutical composition as claimed in claim 1, further comprising a pharmaceutically acceptable carrier.

3. A pharmaceutical composition as claimed in claim 1, wherein the insulin resistance comprises one selected from a group consisting of a type II diabetes, a post-surgical syndrome, a serious illness, a burn injury, a post-traumatic syndrome, a related complication and a combination thereof.

4. A pharmaceutical composition as claimed in claim 1, wherein the far-infrared ray releasing substance irradiates a far-infrared ray with an emissivity over 0.9 at a wavelength in a range of 6-14 μm.

5. A pharmaceutical composition as claimed in claim 1, wherein the far-infrared ray releasing substance reduces the blood glucose at room temperature.

6. A pharmaceutical composition as claimed in claim 1, wherein the far-infrared ray releasing substance further comprises a 1-20% ferric oxide, a 1-10% magnesium oxide and a 1-30% calcium carbonate in weight.

7. A composition for controlling a blood glucose of a subject, comprising:
a far-infrared ray releasing substance,
wherein the composition reduces the blood glucose of the subject via an irradiation of the far-infrared ray releasing substance.

8. A composition as claimed in claim 7, wherein the subject has an insulin resistance.

9. A composition as claimed in claim 7, wherein the far-infrared ray releasing substance comprises an oxide mineral.

10. A composition as claimed in claim 9, wherein the oxide mineral comprises an aluminum oxide.

11. A composition as claimed in claim 7, being used as a pharmaceutical composition.

12. A composition as claimed in claim 11, wherein the pharmaceutical composition further comprises a pharmaceutically acceptable carrier.

13. A composition as claimed in claim 7, further comprising a base material mixed with the far-infrared releasing substance.

14. A composition as claimed in claim 13, wherein the base material is at least one selected from a group consisting of a metal, a glass, a ceramic and a polymer.

15. A method for controlling a blood glucose, comprising the steps of:
providing a far-infrared ray releasing substance; and
disposing the far-infrared ray releasing substance in a place close to a subject in an appropriate distance, wherein the subject has an insulin resistance and the appropriate distance is within an irradiation range of the far infrared ray.

16. A method as claimed in claim 15, wherein the far-infrared ray releasing substance irradiates the far-infrared ray with an emissivity over 0.9 at a wavelength in a range of 6-14 μm, and reduces the blood glucose of the subject via the far-infrared ray irradiation.

17. A method as claimed in claim 15, wherein the far-infrared ray releasing substance comprises an oxide mineral.

18. A method as claimed in claim 17, wherein the oxide mineral comprises an aluminum oxide.

19. A method as claimed in claim 18, wherein the far-infrared ray releasing substance further comprises a 1-20% ferric oxide, a 1-10% magnesium oxide and a 1-30% calcium carbonate in weight.

20. A method as claimed in claim 15, wherein the insulin resistance comprises one selected from a group consisting of a type II diabetes, a post-surgical syndrome, a serious illness, a burn injury, a post-traumatic syndrome, a related complication and a combination thereof.
